(19) United States
(12) Patent Application Publication
(10) Pub. No.: US 2010/0179425 A1
(43) Pub. Date: Jul. 15, 2010

(54) SYSTEMS AND METHODS FOR CONTROLLING ULTRASOUND ENERGY TRANSMITTED THROUGH NON-UNIFORM TISSUE AND COOLING OF SAME

(76) Inventor: Eyal Zadicario, Tel Aviv-Yafo (IL)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/352,932

(22) Filed: Jan. 13, 2009

(51) Int. Cl. A61B 8/00

(52) U.S. Cl. 600/438

(57) ABSTRACT

The emission intensities of transducer elements of an ultrasound transducer array are controlled based on anatomical characteristics of non-uniform tissue regions, e.g., regions of a skull, and a pre-determined threshold.

### FIELD OF THE INVENTION

The field of the invention relates generally to thermal energy treatment systems and, more particularly, to systems and methods for controlling the intensity of acoustic energy transmitted through a non-uniform tissue, such as the skull, and cooling such tissue.

### BACKGROUND

High-intensity focused acoustic waves, such as ultrasound or acoustic waves at a frequency greater than about 20 kilohertz, may be used to therapeutically treat internal tissue regions within a patient. For example, ultrasound waves may be used in applications involving ablation of tumors, thereby eliminating the need for invasive surgery, targeted drug delivery, control of the blood-brain barrier, lysing of clots, and other surgical procedures.

Focused ultrasound systems typically include piezoelectric transducers that are driven by electric signals to produce ultrasound energy. In such systems, a transducer may be geometrically shaped and positioned such that ultrasound energy emitted by an array of transducers collectively forms a focused beam at a “focal zone” corresponding to the target tissue region. As used herein, the terms “beam,” “energy beam,” or “acoustic energy beam” refer generally to the sum of the waves emitted by the various transmitting elements of a focused ultrasound system.

When using a focused ultrasound “energy beam” to thermally treat a certain area of the body, e.g., to ablate a tumor, the beam must be precisely focused to the target location to avoid damage to healthy tissue surrounding the target region. For this purpose, the transducer may be sequentially focused and activated at a number of focal zones in close proximity to one another. For example, this series of “sonications” may be used to cause necrosis of a tissue structure of a desired size and shape.

FIGS. 1 and 2 illustrate a known ultrasound system 100 that may be used for these purposes. The illustrated system 100 includes an imager 110 for determining characteristics of a skull 162 of a patient 160, a phased array 120 of n transducer elements 122, which may be in the form of a spherical cap (as shown in FIG. 2), a controller 140 operably coupled to the imager 110, a signal adjuster 130 operably coupled to the controller 140, and a frequency generator or energy source 150, such as a radio frequency (RF) generator, operably coupled to the signal adjuster 130.

The transducer elements 122 are piezoelectric transducer elements, e.g., piezoelectric ceramic pieces. The signal adjuster 130 includes phase adjustment elements 132₁₋ₙ (generally 132) and associated amplifiers 134₁₋ₙ (generally 134). The frequency generator 150 provides a RF signal as an input to the signal adjuster 130. The RF generator 150 and signal adjuster 130 are configured to drive individual transducer elements 122 of the transducer array 120 at the same frequency, but at different phases. These controls are utilized to transmit ultrasound energy through the patient's skull 162 and to focus the energy at a selected target region within the brain 164. An acoustically conductive fluid or gel 202 is preferably introduced between the inner face of the transducer array 120 and the exterior of the patient's skull 162 in order to prevent any acoustically reflecting air gaps that may reduce the effectiveness of the applied energy.

In the illustrated system 100, n input signals based on the RF generator 150 output are provided to the signal adjuster 130. Coupled to receive each of the n input signals are n pairs of amplifiers 132₁-132ₙ, and associated phase shifters 134₁-134ₙ. Each amplifier 132-phase shifter 134 pair represents a channel of the signal adjuster 130. Phase shifters 134 are configured to provide n independent output signals to the amplifiers 132 by altering or adjusting incoming signals from the RF generator 150 by respective phase shift factors 134. The amplifier 132 outputs drive transducer elements 122, and the collective energy 124 emitted by the transducer elements 122 forms a focused beam of ultrasound energy that traverses the skull 162 and is focused at a target region 210 within the brain 164. Further aspects of known systems 100 and spherical cap transducers are described in U.S. Pat. Nos. 6,612,988 and 6,666,833, the contents of which are incorporated herein by reference as though set forth in full.

While focused ultrasound systems and spherical cap transducers shown in FIGS. 1 and 2 have been used effectively in the past, they can be improved, particularly in procedures involving non-uniform tissue such as the skull. As generally illustrated in FIG. 3, a typical human skull 162 includes multiple tissue layers including an external layer 301, a bone marrow layer 302, and an internal layer or cortex 303, which may be highly irregular in shape. Cortex 303 irregularities may cause certain sections of the skull 162 to be more susceptible to excessive heating when exposed to ultrasound energy. Further, attempts to focus energy at the focal regions 210 may result in excessive heating of certain sections of the skull 162 which, in turn, damages adjacent healthy tissue. Accordingly, by “non-uniform” is meant varying in tissue type, shape and/or conformation so as to respond differently to ultrasound energy.

Known ultrasound therapy systems may operate by focusing an ultrasound beam at a desired focal region 210 with the goal of precisely ablating target tissue. While this avoids ablation of tissue surrounding the target region 212, once again the skull 162 may absorb substantial energy and become heated excessively, resulting in damage to adjacent tissue. One type of injury, in other words, is merely exchanged for another.

### SUMMARY

Embodiments of the invention are directed toward application of focused ultrasound to non-uniform tissue in a manner that avoids harm to healthy anatomy outside the target zone.

In a first aspect, a method for controlling intensities in a transducer array having multiple transducer elements, each being primarily associated with a corresponding tissue region, includes determining anatomical characteristics of non-uniform tissue regions (e.g., the skull) to be traversed when the transducer array delivers focused ultrasound to a target region. For each of the transducer elements, a preferred intensity of ultrasound energy is determined based on the anatomical characteristics of the corresponding non-uniform tissue region and pre-determined energy thresholds (e.g., a maximum temperature) associated with the region. The individual transducer elements are then driven at their respective preferred intensities, thereby directing ultrasound energy through the non-uniform tissue. As a result, the directed ultrasound energy emitted by the transducer array is non-uniform across the transducer array and maximized while satisfying the pre-determined thresholds.

In certain embodiments of the invention, the anatomical characteristics may include the thickness of the non-uniform tissue, the density of the non-uniform tissue, an entrance point of a ray emitted by a transducer element into the non-uniform tissue, and/or an exit point of a ray emitted by a transducer element from the non-uniform tissue. Further, the intensity of the emitted ultrasound energy may also be influenced by an increase in temperature of the non-uniform tissue. In various implementations of the invention, the intensity of ultrasound energy emitted by individual transducer elements may range from 0 Watt to about 10 Watts. The difference between minimum intensity and maximum intensity levels of ultrasound energy emitted by individual transducer elements can vary from 0.0 Watt to about 10 Watts.

In some cases, an actual temperature of the non-uniform tissue is measured (using, for example, magnetic resonance thermometry) and compared to a maximum temperature, and if the measured temperature exceeds the maximum, the non-uniform tissue is cooled. In some instances, the ultrasound transducer may be deactivated.

The cooling process may include circulating a cooling fluid within an interface between the ultrasound transducer and the non-uniform tissue, measuring the temperature of the cooling fluid, comparing the measured temperature to a maximum temperature. An output signal indicating the results of the comparison may be generated and displayed to an operator.

In another aspect, a method for controlling the intensity of ultrasound energy emitted by a transducer array having multiple transducer elements includes determining anatomical characteristics of regions of a non-uniform tissue (such as a skull), simulating, for each transducer element, the effect of heating a corresponding non-uniform tissue region with ultrasound energy using an intensity based on the anatomical characteristics, and determining a maximum intensity of ultrasound energy for each transducer element based on the simulation and a pre-determined threshold (e.g., a maximum temperature).

In some embodiments, an intensity map may be generated based on the simulation that includes ultrasound energy intensity values for each transducer element such that ultrasound energy emitted by the transducer array is maximized and non-uniform across the transducer array while satisfying the pre-determined threshold. The transducer elements may be driven based on the intensity values in order to direct a beam of ultrasound energy through the non-uniform tissue region (e.g., to a target region beyond the non-uniform tissue).

In some cases, the actual temperature of the non-uniform tissue is measured (using, for example, magnetic resonance thermometry) and compared to a maximum temperature, and if the measured temperature exceeds the maximum, the non-uniform tissue is cooled. In some instances, the ultrasound transducer may be deactivated.

The cooling process may include circulating a cooling fluid within an interface between the ultrasound transducer and the non-uniform tissue, measuring the temperature of the cooling fluid, and comparing the measured temperature to a maximum temperature. An output signal indicating the results of the comparison may be generated and displayed to an operator.

In various implementations, the intensity of ultrasound energy emitted by individual transducer elements may range from about 0.0 Watt to about 10 Watts.

In another aspect, a system for controlling an intensity of a transducer array having multiple transducer elements includes an imaging system, a controller and drive circuitry. The imaging system is configured to determine anatomical characteristics of non-uniform tissue regions (e.g., a skull), while the controller is configured to determine a maximum allowable intensity of ultrasound energy emitted by each transducer element into (and through) a corresponding non-uniform tissue region based the determined anatomical characteristics and a pre-determined threshold (such as a maximum temperature) associated with the tissue regions. The drive circuitry drives the transducer elements to emit ultrasound energy at the determined maximum intensities through the non-uniform tissue.

In various embodiments a computed tomography (CT) imaging system may be used to determine the anatomical characteristics of the non-uniform tissue and a magnetic resonance imaging (MRI) system may be used in conjunction with the CT imaging system to localize the transducer elements relative to the non-uniform tissue regions. In certain cases, the MRI system determines an actual temperature of the non-uniform tissue while the transducer elements are being driven, and the controller is further configured to generate an output signal indicating when the measured temperature exceeds the maximum temperature. In some implementations, the individual transducers are independently controllable such that the temperature of each non-uniform tissue region does not exceed the maximum temperature for that region.

The system may also include a fluid interface integrated with the transducer and coupled to the controller such that it is positionable around the non-uniform tissue region and further facilitates the circulation of cooling fluid about the tissue, either periodically or continuously. In some instances, a temperature sensor may be positioned within the interface to allow for the measurement of the cooling fluid and communication of the measured temperature to the controller.

In another aspect, a system for controlling the intensity of a transducer array comprising multiple transducer elements includes an imaging system, a controller and drive circuitry. The imaging system is configured to determine anatomical characteristics of non-uniform tissue regions, and the controller simulates, for each transducer element, the effects of heating corresponding non-uniform tissue regions based at least in part on the determined anatomical characteristics. The controller further determines a maximum allowable intensity of ultrasound energy emitted by each transducer element based on the simulation and a pre-determined threshold, such as a maximum allowable temperature. The drive circuitry causes the transducer elements to emit ultrasound energy at the determined maximum intensities.

In some embodiments, the controller generates an intensity map of ultrasound energy intensity values for each transducer based on the simulation. The system may also include an MRI system that measures the temperature of the non-uniform tissue and based on the temperature, and, if above a maximum temperature, causes the controller to generate an output signal that indicating as such. In some cases, individual transducer elements are independently configurable to ensure that the temperature of each tissue regions does not exceed the maximum temperature.

The system may also include a fluid interface integrated with the transducer and coupled to the controller such that it is positionable around the non-uniform tissue region and further facilitates the circulation of cooling fluid about the tissue, either periodically or continuously. In some instances, a temperature sensor may be positioned within the interface to allow for the measurement of the cooling fluid and communication of the measured temperature to the controller.

In yet another aspect, a method for cooling skull tissue during delivery of ultrasound energy thereto includes positioning the head of a patient within an ultrasound transducer such that a fluid interface integral with the ultrasound transducer is positioned about skull tissue of the patient and between an inner surface of the ultrasound transducer and the skull tissue. Transducer elements are driven in such a manner as to direct a beam of ultrasound energy through the skull tissue, thereby heating the skull tissue, and a cooling fluid is circulated (either periodically or continuously) within the fluid interface to cool the skull. In some cases, the fluid may be circulated prior to delivery of ultrasound energy.

The temperature and/or pressure of the fluid circulating within the interface may be monitored (using, for example, a temperature sensor within the interface) and an output signal indicated whether the fluid has exceeded a maximum temperature may be generated. The signal may be displayed to a user, thereby allowing the user to interrupt the delivery of ultrasound energy to the skull.

In another aspect of the invention, a system for cooling skull tissue of a patient during application of ultrasound energy through the skull tissue includes an ultrasound transducer having multiple transducer elements and a fluid interface. The transducer is positionable about the skull tissue and emits ultrasound energy through the skull tissue. The fluid interface is integral with the ultrasound transducer and positionable between the ultrasound transducer and the skull tissue, and facilitates continuous circulation of cooling fluid about the skull tissue.

### BRIEF DESCRIPTION OF THE DRAWINGS

Referring now to the drawings in which like reference numbers represent corresponding parts throughout and in which:

FIG. 1 is a schematic diagram of an example of a known ultrasound therapy system;

FIG. 2 is a schematic diagram of a known spherical cap transducer that may be used with the ultrasound therapy system shown in FIG. 1;

FIG. 3 generally illustrates tissue layers of a human skull;

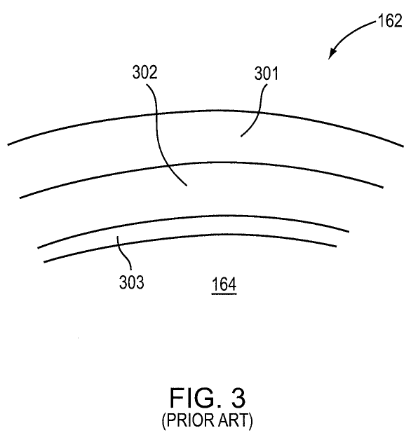

FIG. 4 is a flow chart illustrating a method for controlling the intensity of energy emitted by transducer array elements during therapy involving a non-uniform tissue according to one embodiment of the invention;

FIG. 5 is a flow chart illustrating a method for controlling the intensity of energy emitted by transducer array element during therapy of brain tissue while the temperature of skull tissue remains less than a maximum or threshold temperature according to one embodiment;

FIG. 6 illustrates ray analysis used in embodiments to determine geometric attributes of a skull region;

FIG. 7 is a flow chart illustrating a method of determining intensities involving heating simulations and generation of an intensity map according to one embodiment;

FIG. 8 is a graph illustrating one example of results of thermal simulation conducted according to one embodiment;

FIG. 9 illustrates one example of an intensity map generated according to one embodiment;

FIG. 10 is a flow chart of a method of cooling non-uniform tissue according to one embodiment;

FIG. 11 is a flow chart of a method of cooling non-uniform tissue according to another embodiment in which cooling adjustments are implemented manually or by a controller;

FIG. 12 illustrates a cooling interface constructed according to one embodiment that is integral with an ultrasound transducer and provides for continuous flow of cooling fluid; and

FIG. 13 schematically illustrates a cooling system constructed according to one embodiment that may be utilized with the cooling interface shown in FIG. 12.

### DETAILED DESCRIPTION OF ILLUSTRATED EMBODIMENTS

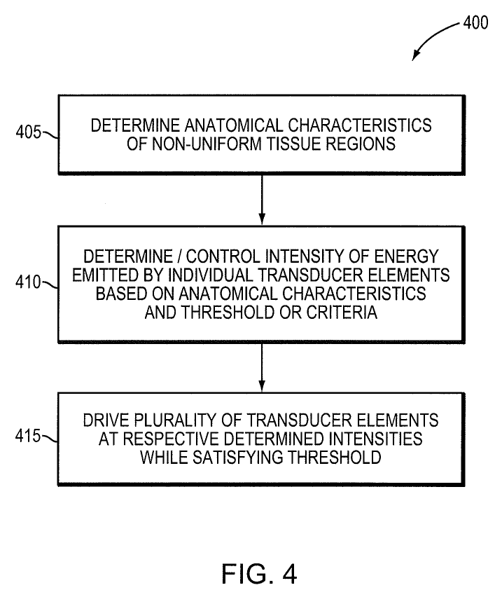

Embodiments of the invention advantageously control and optimize energy emitted by a transducer array to effectively focus energy at a focal zone while maintaining the temperature of non-uniform tissue, such as the skull, at acceptable and safe levels. In particular, embodiments of the invention are capable of precisely focusing an energy beam at a target region to avoid damage to healthy tissue surrounding the target region while also reducing or preventing heating of the skull, thereby also preventing or reducing damage to tissue adjacent to the skull. These significant advantages are achieved by controlling the intensities of energy emitted by individual transducer elements to satisfy skull temperature criteria or thresholds. The expected collective energy may be maximized at the focus, while temperature thresholds or criteria outside the target area are satisfied locally, on an element-by-element basis, and/or globally. A cooling system integral with the transducer may be utilized to monitor the skull tissue temperature and cool the skull tissue as necessary. The cooling system may be used to cool the skull in the event that during therapy, the skull is heated to such a degree such that the skull temperature exceeds a desired or threshold temperature or other safety criterion. Further aspects of embodiments of the invention are described with reference to FIGS. 4-13.

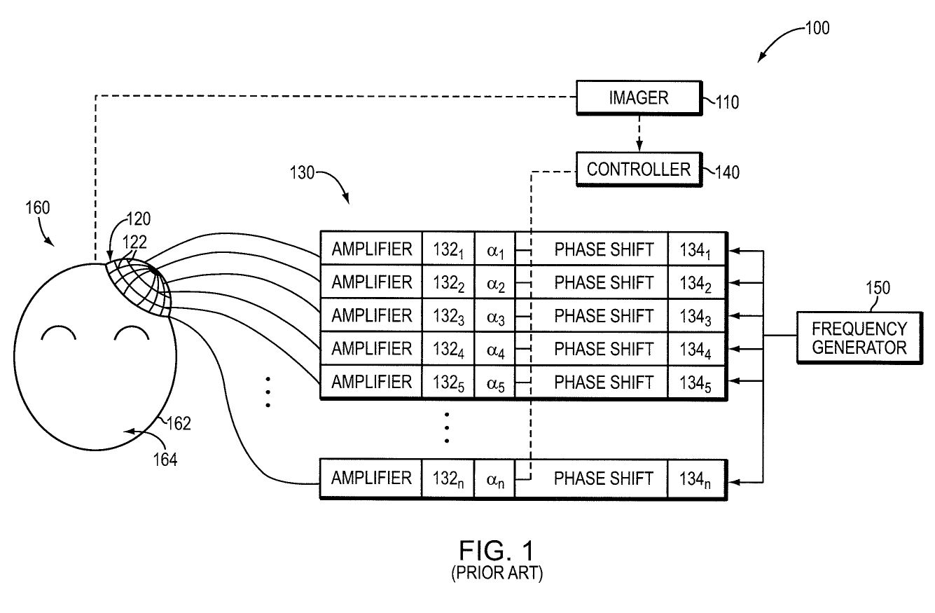

Referring to FIG. 4, a method 400 for controlling the intensity of a transducer array 120 according to one embodiment includes determining anatomical characteristics of non-uniform tissue regions (step 405) using the imager 110 shown in FIG. 1. At step 410, the intensities of individual transducer elements 122 are controlled based on information received from the imager 110, and also, if desired, based on certain pre-determined thresholds or criteria, such as a maximum allowable intensity or other safety criteria. In so doing, the energy intensities 124 emitted by individual transducer elements 122 may be determined and controlled on an element-by-element basis. At step 415, the transducer elements 122 are driven at the respective determined intensities, resulting in a non-uniform intensity distribution across the transducer array 120 and across the non-uniform tissue.

In some embodiments, the transducer elements 122 are driven to generate ultrasound energy 124 at their respective determined intensities while ensuring that the total amount of ultrasound energy delivered collectively satisfies the pre-determined threshold. In particular, the total amount of energy 124 emitted by a transducer array 120 may be selected or maximized by locally maximizing the acoustic energy 124 passing through different skull 162 regions while simultaneously satisfying both the pre-determined threshold on an element-by-element basis and globally across the transducer array 120. As a result, the total ultrasound energy 124 is maximized, focused at the target region 210, and has a non-uniform temperature profile or distribution that satisfies both local (e.g., with respect to individual elements or small groups of elements driven by a single signal) and global thresholds or criteria.

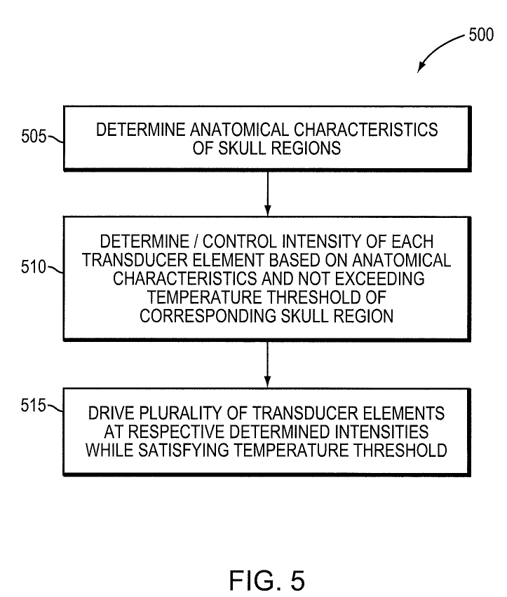

According to one embodiment, the pre-determined threshold is a maximum tissue temperature, and the non-uniform tissue is a skull 162. The skull 162 can be defined as more than one region, each of which may be related to or correspond to a particular transducer element 122 or grouping of elements. Referring specifically to FIG. 5, a method 500 for controlling intensity of energy 124 emitted by a transducer array 120 includes determining anatomical characteristics of multiple regions of a skull 162 (step 505) using imager 110.

In various embodiments (e.g., step 405 of method 400 and/or step 505 of method 500), the imaging system 110 includes computed tomography (CT) imaging and/or magnetic resonance imaging (MRI) elements. CT imaging may be used, for example, to extract anatomical characteristics of the skull 162, such as the skull thickness, local bone densities and/or directional or geometrical features including a normal relative to a surface region of the skull 162. MRI imaging may be used to localize the plurality of transducer elements 122 relative to the skull 162 and/or for purposes of therapy planning. CT and MRI data for a given skull 162 may be combined using multi-modal registration or other similar techniques.

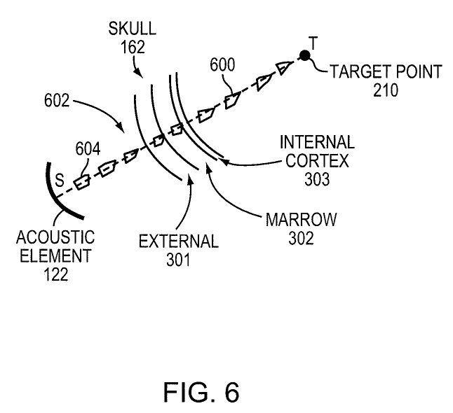

FIG. 6 illustrates a single ray 600 traveling through a voxel of a CT-generated volume representing skull region 602, following placement of transducer elements 122 relative to the skull 162 and target region 210. In some embodiments, a set of x-rays 600 is projected through the CT volume set representing multiple skull regions 602. Pixel values 604 along a ray 600 and extending through each volume or skull region 602 may be determined and arranged to form a CT intensity profile for each skull region 602. The pixel values may represent, for example, the absorption of the x-rays in the skull region 602 (typically measured in “Hounsfield numbers” or “CT numbers”). In some implementations, such information can be used to relate x-ray absorption coefficients with ultrasound absorption coefficients. The CT intensity of bone or skull tissue along each ray 600 is known, and various geometric attributes of a skull region 602 and corresponding rays 600 passing therethrough may be determined based on the CT intensity profile. Examples of such geometric attributes include the entrance point of the ray 600 to the skull region 602, the exit point of the ray 600 from the skull region 602, thicknesses of different skull tissue layers 301-303, and/or an average local density of a CT region 602 in CT units. Data acquired during ray analysis may then be used to construct internal and external surfaces of the skull 162 to create a local geometric characteristic mapping of the skull.

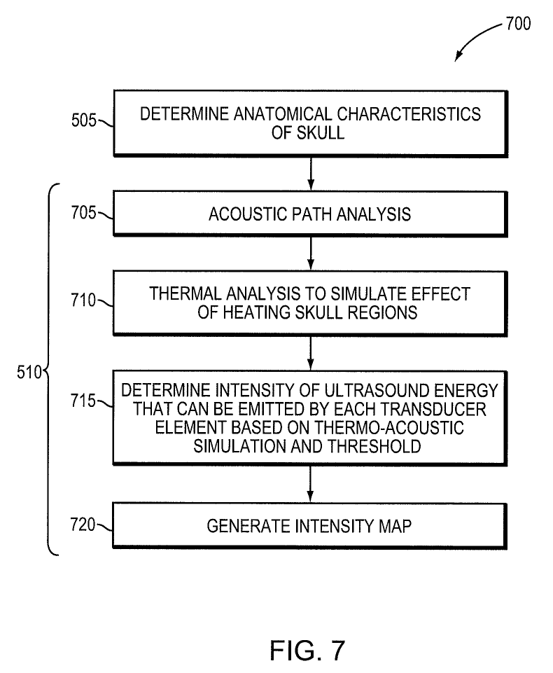

Referring again to FIG. 5, at step 510, the intensity of ultrasound energy 124 emitted by each transducer element 122 may be determined or controlled based on the previously determined anatomical characteristics (step 505) and a maximum or threshold skull or skull region temperature. According to one embodiment, and with further reference to FIG. 7, step 510 may also include a thermo-acoustic simulation. In such cases, the thermo-acoustic simulation can involve analyzing an acoustic path through a skull region 602 (step 705), performing thermal simulations to estimate how different skull regions 602 absorb different quantities of energy and have different heating profiles (step 710), determining the optimal intensity of energy to be emitted by each transducer element 122 (step 715), and generating an intensity map corresponding to transducer elements 122 (step 720). The resulting intensity map includes optimal intensity values of energy emitted by respective individual transducer elements 122, which collectively optimize the energy delivered to a target region 210 while satisfying one or more temperature thresholds or safety criteria as described above.

According to one embodiment, steps 705 and 710 may be performed on an element-by-element basis to estimate how different skull tissue regions 602 will be heated as ultrasound energy 124 traverses the skull 160. For this purpose, the local skull tissue geometry (determined at step 505 and discussed above) and the speed of sound through the skull 600 may be utilized to analyze the acoustic path of ray 600 through skull region 602, and to predict how the skull region 602 will be heated as a result (based on the previously determined anatomical characteristics). In some instances, the speed of sound through the skull region 602 may be determined by utilizing an empirical model that correlates CT density to the speed of sound, or in accordance with other known techniques. A heat equation or model for each skull region 620 may then be solved or applied to predict how a given skull region 602 will be heated by ultrasound energy 124 emitted by a corresponding transducer element 122 or groupings of transducer elements 122.

For example, angles of incidence between a ray 600 and skull 160 surfaces may be analyzed using Snell's law to estimate the path of an acoustic ray 600 emitted by a particular transducer element 122, which traverses the skull region 602 and is directed to a target region 210 in the brain 164. Energy reflected from the skull 160 surface and attenuation and absorption of energy within a skull region 602 can also be estimated utilizing the acoustic path analysis. This analysis may be repeated for each skull region 602 in order to acquire a complete picture of estimated energy reflection, absorption and attenuation for multiple skull regions 602.

Referring again to FIG. 7, acoustic path information (acquired at step 705) is used to simulate how an individual skull region, characterized by the previously performed acoustic path analysis, is heated over time, for each point or pixel 604 along a ray 600 traversing the skull region 602 (step 710). This information may then be used to estimate the amount of energy reflected from the skull 600 and the amount of energy absorbed by the skull, thus impacting heating of the skull region 602.

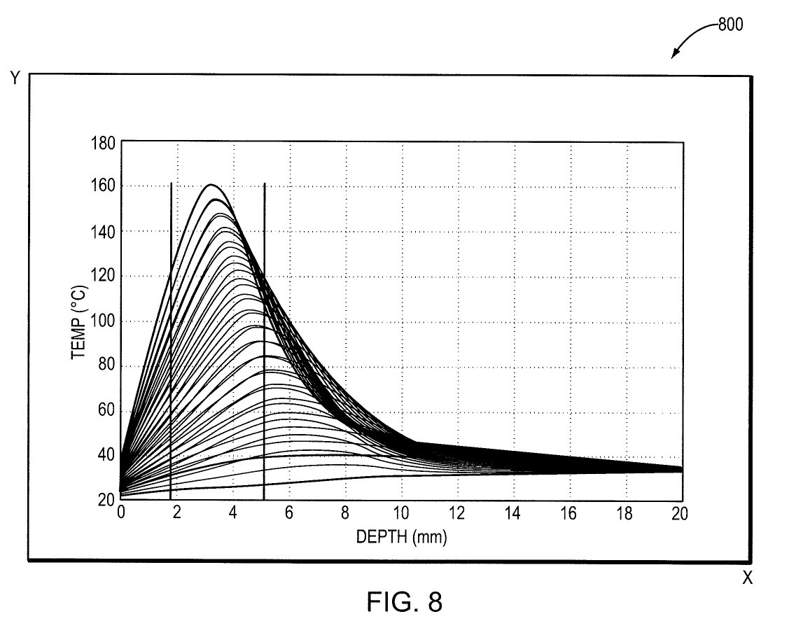

For this purpose, thermal simulations may assume a steady-state temperature profile based on a thermal gradient between the external side 301 of the skull 162, which is cooled by water at a temperature of about 10° C.-20° C., and the tissue distant from the surface at body temperature. A heat expression or model may then be used to iteratively solve heating effects for each skull region. One example of a suitable heat model that may be used for this purpose is a linear heat equation solved numerically with appropriate boundary constraints. The result of thermal simulation for a particular skull region 602 may be expressed as a heat simulation graph 800 (FIG. 8), having skull tissue depth (mm) plotted along the x-axis and simulated temperature increases along the y-axis. The thermal simulation analysis may be conducted for each transducer element 122 (or groupings thereof) and each corresponding skull region 602, thus resulting in a global thermal simulation across the skull 162, and an estimate of the thermal rise of each skull region 602 when exposed to ultrasound energy.

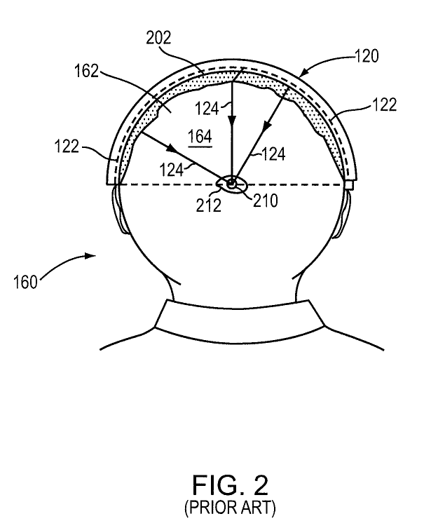

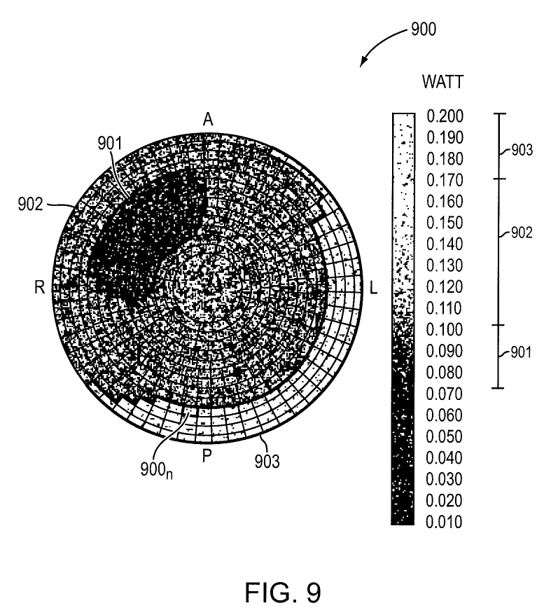

Referring again to FIG. 7, and with further reference to FIG. 9, the optimal or maximum intensity of ultrasound energy 124 to be emitted by each transducer element 122 is determined based on the skull region 602 characteristics and temperature simulation (step 720). According to one embodiment, each skull region 602 may be analyzed to determine the maximum intensity of ultrasound energy 124 that can be absorbed such that the expected temperature rise of the skull region 602 is below a threshold or acceptable maximum temperature. In the illustrated embodiment, the determined or maximum intensity values are collectively represented in the form of an intensity map 900. Each segment 900n of the map 900 represents a transducer element 122 of the transducer array 120, which may be in the form of a spherical cap as represented in FIGS. 2 and 9.

As shown FIG. 9, the intensity values across the transducer array 200 may vary from element to element and are therefore typically non-uniform. For example, regions 901 have a higher heat sensitivity than regions 902 and 903, and region 903 has the lowest heat sensitivity. Different intensity levels may be assigned to certain transducer elements 122 to avoid excessive skull heating. For example, the intensity map 900 dictates that transducer elements 122 corresponding to map section 901 will emit energy 124 at low levels since the corresponding skull regions 602 have the highest heat sensitivity. In contrast, higher intensity ultrasound energy 124 may be applied to other skull regions 602, e.g., skull regions corresponding to map region 903, since these regions are less sensitive to heat generated by ultrasound energy. It should be understood that the identified regions 901-903 are provided for purposes of illustration, and that the change in intensity levels between regions (including neighboring regions) may be gradual or sharp depending on the anatomical structure of corresponding skull regions 602. Moreover, it should be understood that FIG. 9 illustrates one example of an intensity map 900, and that the intensity map 900 may vary depending on different skull structures.

In the illustrated example, transducer elements 122 associated with region 901 are controlled to emit ultrasound energy 124 at about 0.07 to about 0.10 Watt, transducer elements 122 associated with region 902 are controlled to emit ultrasound energy 124 at about 0.10 Watt to about 0.17 Watt, and transducer elements 122 associated with region 903 are controlled to emit ultrasound energy 124 at about 0.17 Watt to about 0.20 Watt. Thus, the power levels range from a minimum value of about 0.07 Watt to a maximum value of about 0.2 Watt, and the difference between minimum and maximum power levels is about 0.13 Watt. In other examples this difference can range from zero to 10 Watts per transducer element.

The intensity of ultrasound energy 124 is selected such that it accommodates the non-uniform tissue structure across skull 162 and forms an optimized, non-uniform intensity distribution, which achieves application of the highest possible level of ultrasound energy to a target region 210 by summation of local energy maxima emitted by individual transducer elements 122 while simultaneously complying with safety criteria such as the temperature of the skull 162 at different regions depending on the underlying characteristics of such skull regions.

By maximizing the overall energy and staying within acceptable energy thresholds, the ultrasound energy 124 actually reaching the focal zone 210 in order to treat the lesion, tumor or clot is also maximized. In this manner, the technique and system facilitate the application of effective therapy by generating a focused beam while at the same time preventing damage to tissue surrounding the target region 21. In cases in which the energy is being directed inside the skull, skull tissue temperature is controlled both locally (based on analysis of tissue non-uniformities), and globally (based on summation of individual elements 122) to satisfy skull temperature thresholds and safety criteria while the collective energy 124 emitted by the plurality of elements 122 is focused.

Thus, embodiments of the present invention function in a novel manner. For example, in typical systems, the intensity of ultrasound energy 124 emitted by transducer elements 122 is adjusted to improve focusing at the target region 210. If a skull region absorbs a substantial amount of energy, resulting in attenuation, such systems may be configured to apply ultrasound energy at even higher intensities to compensate for attenuation in order to maintain or improve focusing. These known control mechanisms, while providing effective focus, may result in further heating of already overheated skull regions 602, thereby causing even more damage to adjacent tissue. In contrast, embodiments of the invention locally control transducer elements 122 such that they apply ultrasound energy 124 to these selected skull regions 602 at lower intensity levels while achieving sufficient focus, thus prioritizing safety over focusing to protect critical or thermally sensitive skull regions 602.

Other embodiments of the invention involve monitoring and controlling the temperatures of skull regions 602 heated by ultrasound energy 124 emitted by transducer elements 122 as described above. While the monitoring and controlling techniques described below may be employed independently of managing the energy emission, the two techniques may also be used in conjunction with each other.

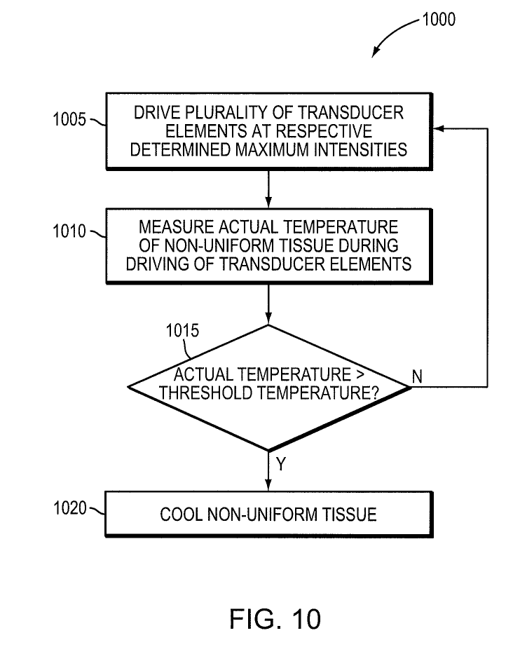

Referring to FIG. 10, a method 1000 of monitoring and controlling a temperature of a skull 162 during ultrasound therapy (step 1005) includes monitoring the actual temperature of the surfaces of one or more skull regions 602 (or, in some cases, the entire skull 162) (step 1010). Step 1005 may, for example be performed while driving the transducer elements 122 according to intensity map 900. In one embodiment, the skull temperature is monitored using magnetic resonance thermometry. The actual temperature of the skull 162 may then be compared to the pre-determined maximum or acceptable temperature (step 1015). According to one embodiment, the maximum temperature of a skull 162 during ultrasound therapy is approximately 107° F., or 42° C. If the actual temperature is below the threshold, therapy can proceed according to the intensity map 900. However, if the actual temperature exceeds the threshold temperature, the skull 162 may be cooled to a safe temperature (step 1020). In addition, the actual temperature readings can be used to calibrate the relationship between applied energy and the resulting tissue temperature, e.g., in creating the intensity map depicted in FIG. 9.

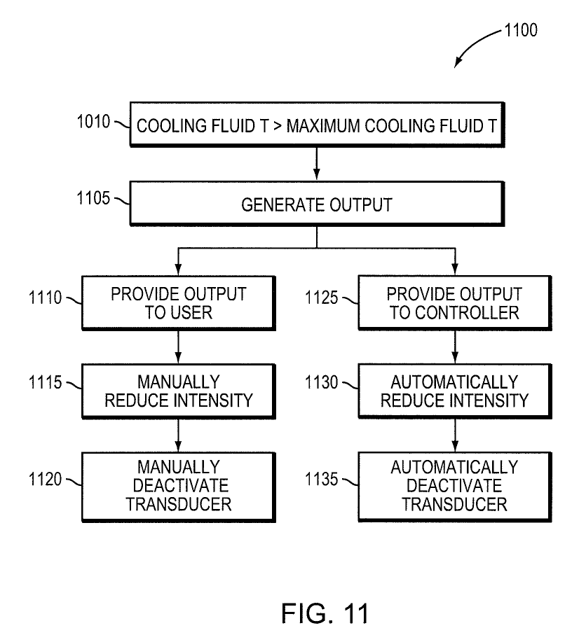

Referring to FIG. 11, according to another embodiment, skull cooling may be implemented by generating an output signal (step 1105) when the temperature of a cooling fluid applied to the skull reaches or exceeds a safety threshold. The output signal may be a visual and/or audible indicator that is provided to an operator via a speaker, display or other device (step 1110). In response to the output signal, the operator may manually reduce the intensity of ultrasound energy 124 (step 1115), by, for example, reducing the intensity of the entire transducer array 120 (and therefore the energy 124 emitted by each transducer element) and/or to only those transducer elements corresponding to thermally sensitive or critical skull regions 620, thereby only affecting the temperatures at these regions. According to another embodiment, the operator may manually deactivate the transducer array 120 (step 1120) to halt sonication altogether. In another embodiment, the generated output is provided to a controller (step 1125), such as a processor, computer or other control element, which may then initiate an automatic reduction in the intensity of ultrasound energy 124 when the temperature of the cooling fluid reaches or exceeds the threshold. Such reductions may include reducing the energy emitted by all of the transducer elements, thereby ensuring reduction in the intensity of energy 124 reaching thermally sensitive or critical skull regions 602, and/or automatically deactivating the transducer array 120 altogether to halt sonication.

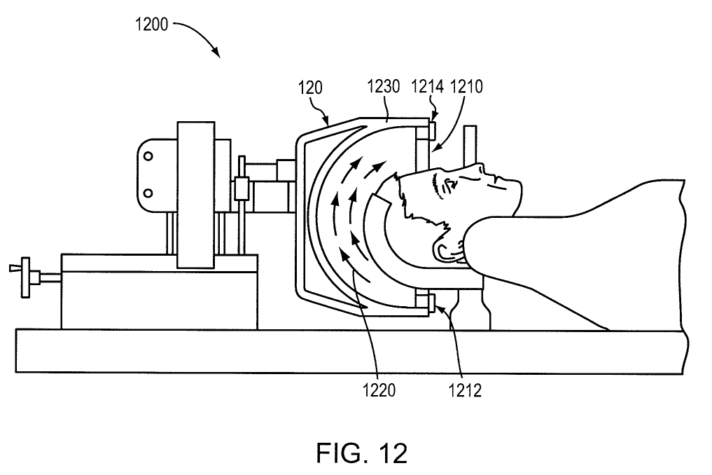

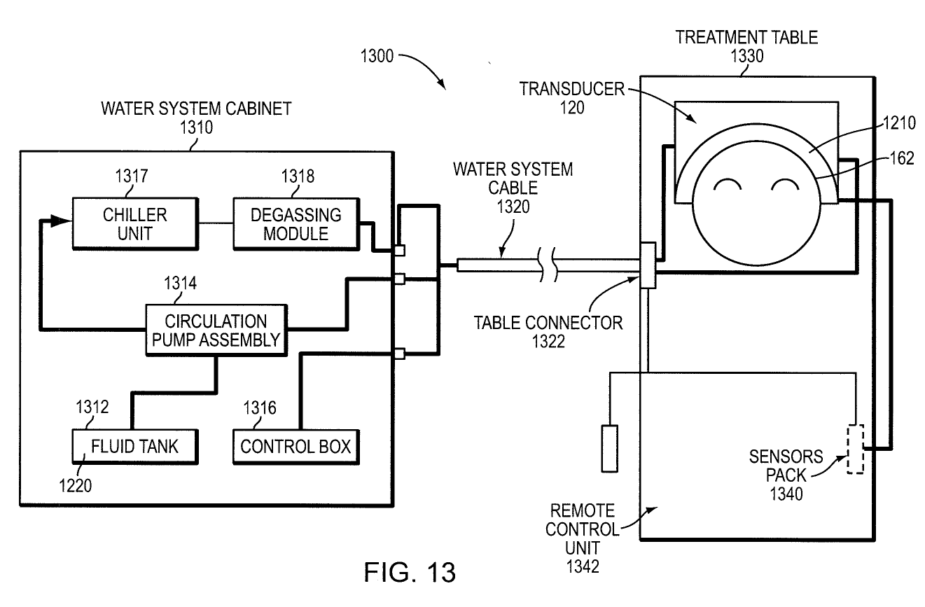

Skull cooling may be achieved by employing a cooling element integral with the ultrasound transducer array 120. The cooling element may be manually or automatically controlled. Referring to FIGS. 12 and 13, the integrated cooling element 1200 may include a fluid interface 1202 that is integral with, or attached to, the ultrasound transducer array 120 and positioned between the transducer array 120 and the patient's skull 162. The interface 1202 is preferably made of a compliant and flexible material to facilitate positioning around the skull and adjustment as necessary to provide a tight interface. Cooling fluid 1220 continuously circulates within or flows through the interface 1210 via a fluid inlet 1212, exiting the interface 1210 via a fluid outlet 1214 or re-circulating as desired. By continuously circulating cooling fluid 1220 through the interface 1202, the skull (or other tissue) can be kept below a ceiling temperature during administration of ultrasound.

According to one embodiment, the cooling interface 1210 is controlled based on the temperature of the skull 162, which may be determined by an external sensor or device, including, for example, magnetic resonance thermometry as described above. According to another embodiment, the temperature of the cooling fluid rather than the skull is measured from within the interface 1210, e.g., using an internal temperature sensor 1230 positioned inside the interface 1210 and within the flow path of the fluid 1220 such that fluid 1220 flows through or about the temperature sensor 1230. The temperature of the cooling fluid 1220 may be monitored to determine if a pre-determined threshold or maximum fluid 1220 temperature has been reached, indicating that the skull temperature is too high. Appropriate action may then be taken in response to these elevated temperatures, including supplying additional cooling fluid 1220, reducing the temperature of the cooling fluid 1220, and/or increasing the flow rate of cooling fluid 1220.

FIG. 13 illustrates one example of a cooling system 1300 that may be used for circulation or flow of cooling fluid 1220 through an integrated cooling element or interface 1210 as shown in FIG. 12. The illustrated system 1300 includes a cabinet 1310 having a source of fluid 1312, which supplies cooling fluid 1220 to a circulation pump 1314. A controller 1316 controls the pump 1314 to circulate fluid 1220 through a chiller unit 1317, and chilled fluid 1220 is degassed 1318 and provided to the inlet 1212 of the integrated cooling element 1210 through a suitable conduit 1320 and connector 1322 that interfaces with the transducer array 120 and treatment table 1330. One or more sensors 1340, whether external sensors on the patient's skull or internal sensors positioned within the cooling interface 1210, are provided to monitor, determine or estimate the temperature of the skull 162. Temperature data can be transmitted to a controller 1316 wirelessly or via a remote control unit 1342 (or other suitable device operably connected to or in communication with the controller 1316), and the controller 1316 may implement appropriate adjustments to the output of transducer array 120 as necessary to achieve or maintain a target skull temperature.

Although particular embodiments have been shown and described, it should be understood that the above description is not intended to limit the scope of embodiments since various changes and modifications may be made without departing from the scope of the claims. It should be understood that embodiments directed to controlling the intensity of energy emitted by a transducer on an element-by-element or local basis may be utilized independently of or in conjunction with other embodiments. Further, although embodiments are described in applications involving transmission of ultrasound energy through skull tissue, embodiments may also be applicable in other treatments involving other non-uniform types of tissue. Moreover, although the advantages of embodiments are most readily realized by controlling the intensity of energy on an element-by-element basis, embodiments may also be configured in other ways that achieve similar results. For example, embodiments may be configured for control of intensity of energy emitted by pairs or other groupings of multiple ultrasound elements. Further, although certain figures illustrate one examples of an intensity map that may be used with one particular skull, it should be understood that the distribution, intensity levels and intensity difference may vary depending on, for example, the configuration of a subject skull. Thus, embodiments are intended to cover alternatives, modifications, and equivalents that fall within the scope of the claims.

What is claimed is:

1. A method for controlling intensities in a transducer array comprising a plurality of transducer elements, the method comprising:
determining anatomical characteristics of non-uniform tissue regions to be traversed using focused ultrasound, each of the transducer elements being primarily associated with a corresponding tissue region;
determining, for each of the transducer elements, a preferred intensity of ultrasound energy at a target region, the intensity being based on anatomical characteristics of the corresponding non-uniform tissue region and a pre-determined energy threshold associated therewith; and
driving the transducer elements at their respective preferred intensities, thereby directing ultrasound energy through the non-uniform tissue to the focus, the directed ultrasound energy emitted by the transducer array being non-uniform across the transducer array and maximized while satisfying the pre-determined thresholds.

2. The method of claim 1, wherein the anatomical characteristics comprise at least one of a thickness of the non-uniform tissue, a density of the non-uniform tissue, a density distribution, an entrance point of a ray emitted by a transducer element into the non-uniform tissue, or an exit point of a ray emitted by the transducer element from the non-uniform tissue.

3. The method of claim 1, wherein the pre-determined threshold is a maximum temperature.

4. The method of claim 3, wherein the intensity of ultrasound energy emitted by the plurality of transducer elements is further based at least in part on an increase in temperature of the non-uniform tissue region.

5. The method of claim 3, further comprising:
determining an actual temperature of the non-uniform tissue during driving of the transducer array;
comparing the actual temperature to the maximum temperature; and
cooling the non-uniform tissue if the actual temperature is greater than the maximum temperature.

6. The method of claim 5, wherein the actual temperature is determined by magnetic resonance thermometry, and further comprising deactivating the ultrasound transducer if the actual temperature is greater than the maximum temperature.

7. The method of claim 3, further comprising
circulating a cooling fluid within a fluid interface integral with the ultrasound transducer and positioned between the ultrasound transducer and the non-uniform tissue;
measuring a temperature of the cooling fluid;
comparing the measured temperature to a maximum cooling fluid temperature; and
generating an output signal representing the results of the comparison.

8. The method of claim 1 wherein the non-uniform tissue comprises skull tissue.

9. The method of claim 7, wherein the output signal is displayed to an operator of the transducer array.

10. The method of claim 7, wherein the cooling fluid circulates within the interface.

11. The method of claim 1, wherein the intensity of ultrasound energy emitted by individual transducer elements ranges from about 0.0 Watt to about 10 Watts.

12. The method of claim 1, wherein a difference between a minimum intensity and a maximum intensity of ultrasound energy emitted by individual transducer elements varies by about 0.0 Watt to about 10 Watts.

13. A system for controlling an intensity of a transducer array having a plurality of transducer elements, the system comprising:
an imaging system configured to determine anatomical characteristics of non-uniform tissue regions;
a controller operably, coupled to the imaging system, for determining a maximum allowable intensity of ultrasound energy emitted by each transducer element into a corresponding non-uniform tissue region based the determined anatomical characteristics and a pre-determined threshold associated with the tissue regions; and
drive circuitry for driving the transducer elements to emit ultrasound energy at the determined maximum intensities through the non-uniform tissue.

14. The system of claim 13 further comprising a computed tomography imaging system for determining the anatomical characteristics of the non-uniform tissue regions.

15. The system of claim 14 further comprising a magnetic resonance imaging system, associated with the computed tomography system, for localizing the plurality of transducer elements relative to the non-uniform tissue regions.

16. The system of claim 13, wherein the predetermined threshold is a maximum temperature of a non-uniform tissue region.

17. The system if claim 13 wherein the non-uniform tissue regions comprise skull tissue.

18. The system of claim 16, wherein the magnetic resonance imaging system is configured to determine an actual temperature of the non-uniform tissue during driving of the plurality of transducer elements, the controller being configured to generate an output indicating when the actual temperature of the non-uniform tissue is greater than the maximum temperature.

19. The system of claim 16, wherein individual transducer elements are independently controllable such that the temperature of each non-uniform tissue region does not exceed the maximum temperature.

20. The system of claim 13, further comprising a fluid interface integral with the ultrasound transducer and operably coupled to the controller, wherein the fluid interface is positionable around the non-uniform tissue region and configured to facilitate circulation of cooling fluid about the non-uniform tissue.

21. The system of claim 20, further comprising a temperature sensor positioned within the fluid interface and in communication with the controller, the temperature sensor being configured to measure a temperature of the cooling fluid circulating within the fluid interface.

22. The system of claim 21, wherein the fluid circulates within the interface in a continuous fashion.
